(19) United States
(12) Patent Application Publication
(10) Pub. No.: US 2005/0026209 A1
(43) Pub. Date: Feb. 3, 2005

(54) Optical fiber bundle for detecting binding of chemical species

(76) Inventor: Charles S. Vann, Burlingame, CA (US)

Correspondence Address: MORGAN, LEWIS & BOCKIUS, LLP., 2 PALO ALTO SQUARE, 3000 EL CAMINO REAL, PALO ALTO, CA, 94306, US

(21) Appl. No.: 10/932,608

(22) Filed: Sep. 1, 2004

Related U.S. Application Data
(63) Continuation-in-part of application No. 10/877,113, filed on Jun. 24, 2004.
Application No. 10/877,113 is a continuation-in-part of application No. 10/602,900, filed on Jun. 23, 2003.
Application No. 10/602,900 is a division of application No. 09/590,761, filed on Jun. 8, 2000, now Pat. No. 6,649,404.
Application No. 09/590,761 is a division of application No. 09/479,181, filed on Jan. 7, 2000, now Pat. No. 6,635,470.
Application No. 09/479,181 is a continuation-in-part of application No. 09/227,799, filed on Jan. 8, 1999, now abandoned.

(51) Int. Cl. C12Q 1/68

(52) U.S. Cl. 435/6; 385/115

(57) ABSTRACT

The system includes a bundle of elongate optical fibers, multiple probes, a well, a light source and a detector. The optical fibers each have a first end remote from a second end. Each of the multiple probes is attached to one of the optical fibers within a predetermined section between each of the optical fiber's first and second ends. The well is configured to hold a solution comprising a target and to receive at least the predetermined section of each of the optical fibers. The light source is configured to direct light into the first end of each of the optical fibers. Finally, the detector is configured to detect light emitted by the binding of the target to at least one of the multiple probes. In some embodiments, there are multiple bundles and multiple wells.

### PRIORITY AND RELATED APPLICATIONS

This application is a continuation-in-part of prior U.S. application Ser. No. 10/877,113, filed Jun. 24, 2004, which application is a continuation-in-part of prior U.S. application Ser. No. 10/602,900,filed Jun. 23, 2003, which application is a divisional application of U.S. application Ser. No. 09/590,761, filed Jun. 8, 2000, now U.S. Pat. No. 6,649,404, issued Nov. 18, 2003, which application is a divisional of U.S. application Ser. No. 09/479,181, filed Jan. 7, 2000, now U.S. Pat. No. 6,635,470, issued Oct. 21, 2003 which application is a continuation-in-part application of U.S. application Ser. No. 09/227,799, filed Jan. 8, 1999, now abandoned, all of which applications and patents are incorporated herein by reference in their entirety.

### FIELD

The invention relates generally to the detection of contact or binding of chemical species.

### INTRODUCTION

Presently, DNA micro-arrays or DNA (gene) chips are used for a wide range of applications such as gene discovery, disease diagnosis, drug discovery (pharmacogenomics) and toxicological research (toxicogenomics). Typically, an array of immobilized chemical compounds or probes are contacted with a target of interest, and those compounds in the array that bind to the target are identified. Existing methods for manufacturing these micro-arrays generally include: 1) in-situ methods where multiple compounds are synthesized directly onto a substrate to form a high density micro-array or 2) deposition methods in which pre-synthesized compounds are covalently attached to the surface of the substrate at appropriate spatial addresses by sophisticated robot dispensing devices. However, the in-situ method typically requires specialized reagents and complex masking strategies, and the deposition method typically requires complex robotic delivery of precise quantities of reagents. Furthermore, bead-based assay systems typically require redundancy to obtain a useful result. For example, over 1000 beads may be required to identify only 10 binding sites. Such systems also require a complicated decoding step, as the location of each bead is not tracked during the analysis process. Accordingly, existing methods for manufacturing micro-arrays are complex and expensive. As a result, there is a need for a simple and cost-effective high-throughput system and method for detecting the binding of chemical species.

### SUMMARY

In one embodiment, a system for detecting binding of two chemical species is provided. The system includes a bundle of elongate optical fibers, multiple probes, a well, a light source and a detector. The optical fibers each have a first end remote from a second end. Each of the multiple probes is attached to one of the optical fibers within a predetermined section between each of the optical fiber's first and second ends. The well is configured to hold a solution comprising a target and to receive at least the predetermined section of each of the optical fibers. The light source is configured to direct light into the first end of each of the optical fibers. Finally, the detector is configured to detect light emitted by the binding of the target to at least one of the multiple probes. In some embodiments, there are multiple bundles and multiple wells.

A method for detecting binding of two chemical species is also provided. A target is contacted with multiple probes each attached to a different elongated optical fiber of a bundle of elongated optical fibers between a first end and a second end of each of the optical fibers. Light is then directed at the first end of each of the optical fibers. Light emitted by the binding of the target to at least one of the multiple probes is then detected near the second end of each of the optical fibers.

Also provided is a method for making an optical fiber having known probes attached thereto. A known sequence is provided in a solution. A first probe having a zip code sequence (TSO-Zip) attached thereto is then inserted into the solution. A second probe that is labeled and ligation enzymes are also inserted into the solution. The first and second probes then hybridize with the known sequence. The first and second probes then covalently bond to each other using a ligation enzyme of the ligation enzymes to form a ligated probe sequence. The ligated probe sequence is removed from the known sequence. A fiber is inserted into the solution. The fiber has a third probe attached thereto. A Zip template is added into the solution. The Zip template is configured to hybridize to both the TSO-Zip and the third probe. The TSO-Zip and the third probe covalently bond to each another using a ligation enzyme of the ligation enzymes. The TSO-Zip and the third probe are then removed from the Zip template sequence.

Another method for making an optical fiber having known probes attached thereto provides a known sequence in a solution. A first probe is inserted into the solution. The first probe has a zip code sequence (TSO-Zip) attached thereto and a sequence for hybridization with a universal forward primer attached to the TSO-Zip. A second probe is added into the solution, wherein the second probe is attached to a sequence for hybridization with a universal reverse primer. A forward primer is also added to the solution. A reverse primer is also to the solution, wherein the reverse primer is labeled. A polymerase is then added to the solution. The first and second probes hybridize with the known sequence. Ligation enzymes are added into the solution, whereafter the first and second probes covalently bond to each other using a ligation enzyme of the ligation enzymes to form a ligated probe sequence. The ligated probe sequence is removed from the known sequence. The ligated probe sequence is them amplified using the forward primer, the reverse primer, the polymerase and the ligated probe sequence through a polymerase chain reaction (PCR) technique. A fiber is then inserted into the solution, wherein the fiber has a third probe attached thereto. The TSO-Zip and the third probe then hybridize to one another.

Yet another method for making an optical fiber having known probes attached thereto, provides a known sequence in a solution and inserts a first probe into the solution. At least part of the first probe has a sequence that will hybridize with a portion of the known sequence. The probe has a biotin attached thereto. Ligation enzymes are added into the solution. Thereafter, a fiber is inserted into the solution. The fiber has a second probe attached thereto. At least part of the second probe has a sequence that will hybridize with a portion of the known sequence. The first probe and the second probe are allowed to hybridize to the known target. The first and second probes are allowed to covalently bond to each other using a ligation enzyme of the ligation enzymes to form a ligated probe sequence. The ligated probe sequence is then removed from the known sequence, and a label attached to the biotin.

Accordingly, the invention provides a multi-functional detection system capable of Single Nucleotide Polymorphisms (SNPs) or gene expression and analysis. No decoding is required, as is the case with bead-based assay systems. This is because the location of each fiber, and hence the location of each probe, is known, i.e., the system never looses track of the fiber as is the case in bead-based assay systems. Furthermore, the present invention does not require any redundancy, as is the case with bead-based assay systems.

Each fiber array bundle provides a high density of fibers. For example, the present invention is able to bundle 6600 fibers together into a single compact bundle, where each fiber can have multiple probes attached thereto. For example, four different probes are attached to each of the 6600 fibers and used in combination with a standard 96 well microtiter plate. This provides a mechanism for testing to more than 2.5 million distinct Single Nucleotide Polymorphisms (SNPs).

The present invention also provides for high-throughput detection. For example, detection may take a fraction of a second for each optical fiber, thereby requiring less than one hour read-time per 96-well mictotiter plate.

Furthermore, in some embodiments, the detector can detect a large range of light emitted from the fibers, i.e., from a dim light to a bright light without being saturated. For example, the detector may have a 10⁴ dynamic range of detection, where the dynamic range is the detection ability of the detector, which translates into the range of targets that can be reliably detected.

In addition, some embodiments provide for real-time analysis, where homogeneous assays are possible. For example, once the target is placed into a well containing the fibers, the well does not need to be emptied for the analysis to proceed.

It is also possible to make probes universal, as described below. This has the added advantage of reducing the overall load on quality control, as fewer fibers and probes need to be checked.

Through the use of fibers, the fiber arrays of the invention provide many advantages over currently available micro-arrays. For example, fibers having a chemical species immobilized thereon can be prepared in advance and stored, thereby permitting rapid assembly of customized arrays.

Moreover, the invention provides reliability that is presently unattainable in the art. For the conventional systems described above, verifying the integrity of an array prior to use is virtually impossible—chemical species immobilized at each spot in the array would have to be individually analyzed—a task which is labor intensive and, given the small quantities of chemical species immobilized at a spot, may even be impossible. In the instant invention, the integrity of the chemical species immobilized on a fiber can be determined by simply analyzing a section of the optical fiber.

Because the chemistry for fabricating the fibers can be performed in advance, the present invention also avoids wicking, cleaning, and on-line loading associated with immobilizing the chemical using current deposition methods, such as in-situ methods. In-situ methods also require the development of specialized chemistries and/or masking strategies. Spotting a micro-array, requires the handling of thousands of drops that have to be placed in very specific locations defined by two dimensions. Furthermore, spotting may result in contamination between adjacent contact points. In contrast, the present invention does not suffer from these drawbacks and takes advantage of well-known chemistries, which do not require deposition of precise volumes of liquids at defined xy-coordinates. Fibers, each having different chemical species immobilized thereon, may be placed next to each other with a reduced potential for such contamination with an adjacent fiber.

Use of the fiber array of the present invention also allows the mobile chemical species or target solution to be easily dispensed into wells for subsequent contact with the fibers. In addition, different target solutions may be dispensed into separate wells, which allows the contact between each probe-target pair to be unique. Furthermore, the present invention provides for a relatively high signal to noise ratio, since the use of fibers with optical properties allows for more controlled illumination. Also, there is no, or very little, signal cross-talk between adjacent fibers.

The fiber array of the present invention is well suited for use in performing nucleic hybridization assays for applications such as sequencing by hybridization and detecting polymorphisms among others.

Accordingly, the present invention is simpler, less complex and less costly to manufacture and use then current systems and methods.

These and other features of the present teachings are set forth herein.

### DRAWINGS

The skilled artisan will understand that the drawings, described below, are for illustration purposes only. The drawings are not intended to limit the scope of the present teachings in any way.

FIG. 1 is a partial cross-sectional oblique view of a portion of a system for detecting the binding of chemical species according to an embodiment of the invention;

FIG. 2 is a partial cross-sectional oblique view of the system for detecting the binding of chemical species shown in FIG. 1;

FIG. 3A is a perspective view of a partially assembled bundle of optical fibers to be used in the system shown in FIGS. 1 and 2;

FIG. 3B is a perspective view of the assembled bundle shown in FIG. 3A;

FIG. 4 is another embodiment of two rows of a bundle according to another embodiment of the invention;

FIG. 5 is yet another embodiment of a bundle according to yet another embodiment of the invention;

FIGS. 6A-6D are a flow chart of different methods for attaching probes to optical fibers and detecting binding of a probe with a target according to an embodiment of the invention;

FIGS. 7A-7D are oblique views of a system and method for making an array according to an embodiment of the invention;

FIGS. 8A and 8B are oblique views of another system and method for making an array according to another embodiment of the invention;

FIGS. 9A-9D are oblique views of yet another system for making an array according to yet another embodiment of the invention;

FIG. 10 is an oblique view of another system for detecting the binding of chemical species according to yet another embodiment of the invention;

FIG. 11 is a side view of another system for detecting the binding of chemical species according to another embodiment of the invention; and

FIG. 12 is a flow chart of a method for detecting the binding of chemical species according to an embodiment of the invention.

### DESCRIPTION OF VARIOUS EMBODIMENTS

The system and method of the present invention provides a simple and reliable system for detecting the binding of at least two chemical species. For a better understanding of the nature of the invention, reference should be made to the following detailed description, taken in conjunction with the accompanying drawings. Like reference numerals refer to corresponding parts throughout the several views of the drawings. Furthermore, aspects of the present teachings may be further understood in light of the examples described below, which should not be construed as limiting the scope of the present teachings in any way.

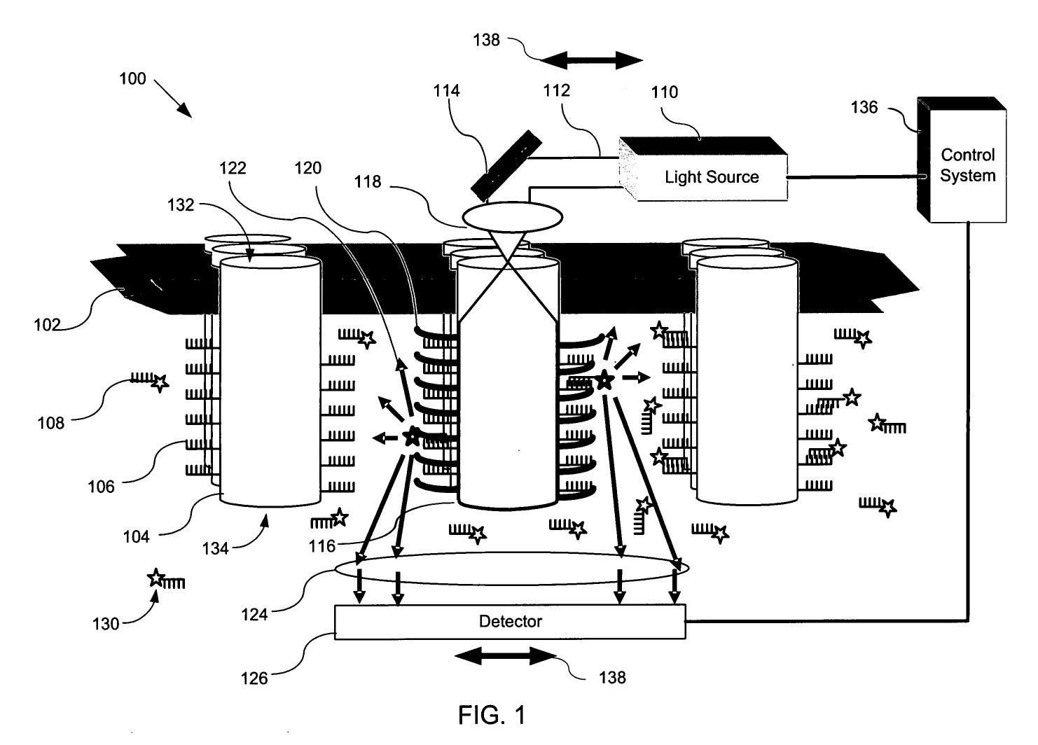

FIG. 1 is a partial cross-sectional oblique view of a portion of a system 100 for detecting the binding of a mobile chemical species 108 to an immobilized chemical species 106. The system 100 includes a support 102 to which multiple substantially parallel elongate optical fibers 104 are coupled. For purposes of the present embodiment, an optical fiber is any material used as a fiber that is transparent to a given wavelength or wavelengths of light. Suitable optical fibers have a diameter of approximately 50 μm to approximately 500 μm, and more preferably 150 μm to 500 μm. Each group of multiple elongate optical fibers 104 will be referred to herein as a bundle or a bundle of fibers. The dimensions of a bundle are preferably small. For example, a bundle with two thousand 50 μm diameter fibers at a 100 μm pitch between fibers would have a cross section of 4.5 mm×4.5 mm. In addition, the length of a fiber bundle should be sufficient for ease of handling, e.g., at least 20 mm long.

The support 102 serves to couple a bundle of fibers to one another and maintain the orientation of the fibers substantially parallel to one another, particularly near the optical fibers″ends. The support 102 also serves to keep the optical fibers from contacting one another, thereby avoiding cross contamination of chemical species and transfer of light between fibers. In some embodiments the support is opaque, while in other embodiments the support is made from a reflective material. The support may also be made from a transparent material.

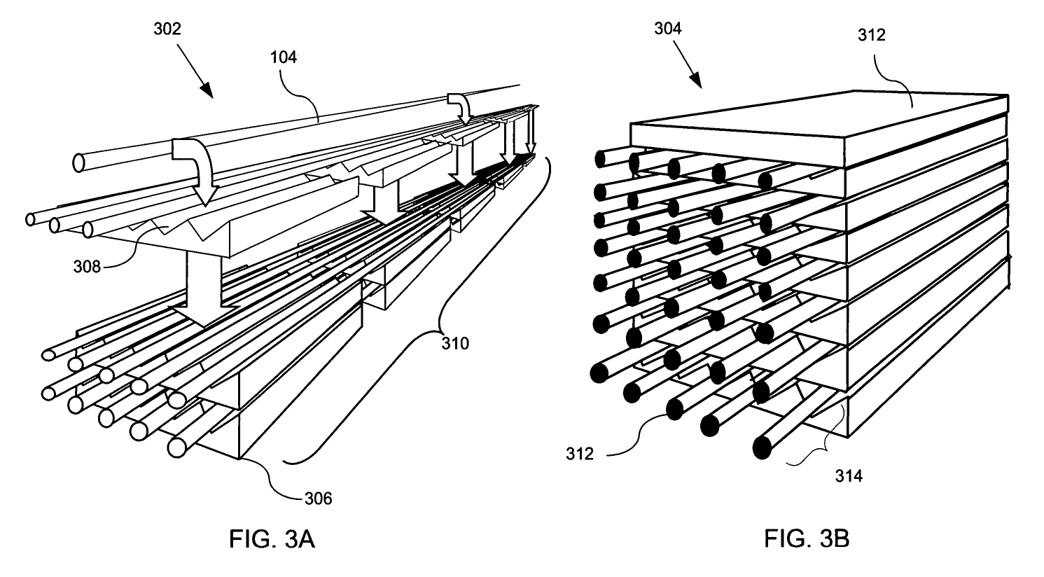

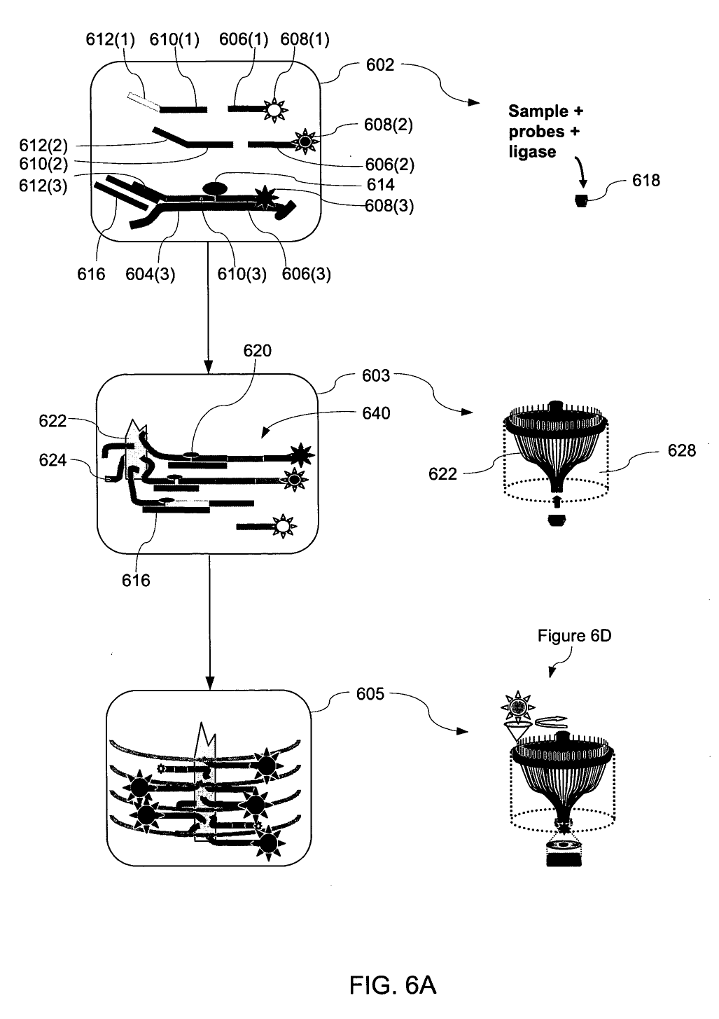

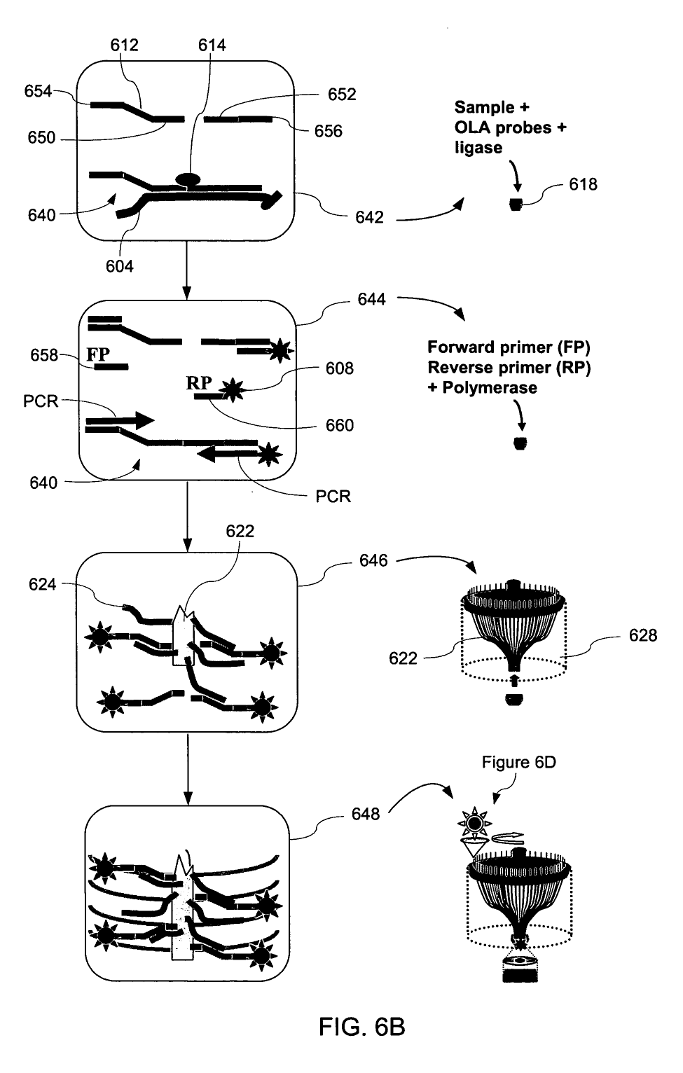

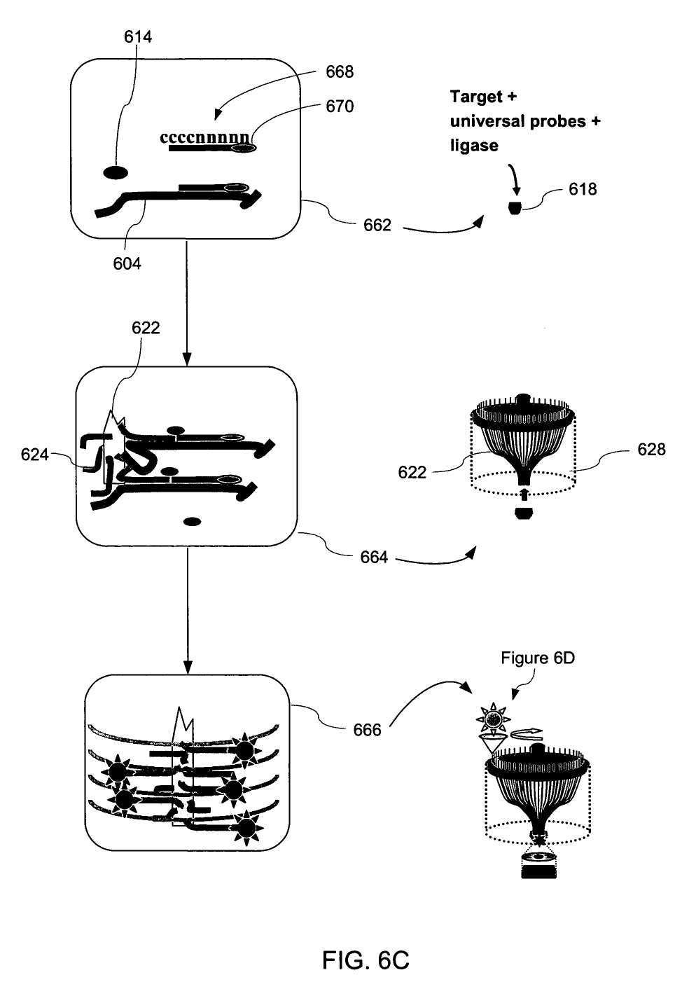

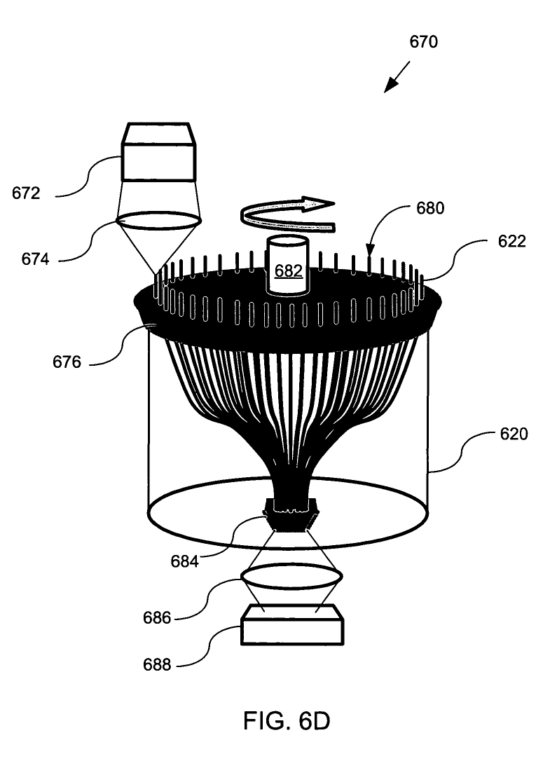

Each fiber 104 has a first end 132 and a second end 134 remote from the first end 132. The second end 134 may be coated with a reflective coating (see FIG. 3B), such as a metal like silver or gold, to prevent light from exiting the fiber at the second end, which may damage a detector 126 and/or provide false readings. In some embodiments each fiber has a different chemical species or probe attached to the outer surface thereof, i.e., the immobilized chemical species 106 is attached to the outer surface of each fiber between the first and the second ends 132, 134, respectively. In some embodiments, the immobilized chemical species 106 is an oligonucleotide probe. Alternatively, different types of probes may be attached to the fiber, and any probe may be attached at different sections or lengths along the fiber. Yet other embodiments, as described below with respect to FIGS. 6A-6D, illustrate multiple, different probes attached to each fiber in a random arrangement. The fibers may have any suitable cross-section, such as a circular or square cross-section. Furthermore, each immobilized chemical species 106 may be attached to a fiber using any suitable method, such as those described in relation to FIGS. 32-44 of U.S. Pat. No. 6,573,089 entitled “method for using and making a fiber array,” which is incorporated herein by reference in its entirety.

Either the immobilized chemical species (e.g. probe) 106 or the mobile chemical species (e.g. target) 108 is labeled. In some embodiments, the mobile chemical species or target 108 is labeled with a moiety 130 that produces a detectable signal when excited by light. However, it should be appreciated that any label capable of producing a detectable signal can be used. Such labels include, but are not limited to, radioisotopes, chromophores, fluorophores, lumophores, chemiluminescent moieties, etc. The label may also be a compound capable of indirectly producing a detectable signal, such as an enzyme capable of catalyzing, e.g., a light-emitting reaction or a colorimetric reaction. The label may also be a moiety capable of absorbing or emitting light, such as a chromophore or a fluorophore.

Alternatively, both chemical species (target and probe) may be unlabeled, and their interaction is indirectly analyzed with a reporter moiety that specifically detects the interaction. For example, binding between an immobilized antigen and a first antibody (or visa versa) could be analyzed with a labeled second antibody specific for the antigen-first antibody complex. For polynucleic acids, the presence of hybrids could be detected by intercalating dyes, such as ethidium bromide, which are specific for double-stranded nucleic acids.

The system 100 further includes a detection apparatus having a light source 110; various optical elements 114, 118 and 124; a detector 126; and a control system 136. The light source 110 may be an excitation laser or an arc lamp that produces a beam of light having the desired wavelength, such as a 532-nm diode-laser with an output of about 3 milliwatts. The optical elements may include a scanning mirror 114 and a focusing lens 118 for redirecting and focusing light produced by the light source 110 at a first end 132 of a fiber 104. The optical elements may also include one or more additional focusing lenses 124 for collecting photons generated by light emitted as a result of binding between the chemical species and for focusing such photons into the detector 126.

The optical elements may also include a light guide and a low-pass filter (not shown). The low-pass filter may be a KV low-pass filter coupled into the light guide that absorbs any excitation light while transmitting the fluorescent light. The KV filter has a very low fluorescence and is angle insensitive. The KV filter is thinned to allow it to fit into the detector's housing, thereby reducing excitation light by about seven orders of magnitude. In one embodiment, the light guide is a one-inch long, 850 μm diameter fused silica rod with a highly smoothed face at the collection end. A light absorbing coating prevents light from entering off-axis. The numerical aperture is preferably 0.5 (55 degrees), thereby accepting a very high theoretical percentage of light from a relatively small fiber over a reflective surface, which may be about 43%.

The detector 126 is a light detecting device that is configured to accurately detect photons that have been generated as a result of binding between the chemical species, e.g., target and probe. A suitable detector 126 may be a photon multiplier tube (PMT) assembly. The PMT assembly may consist of a HAMAMATSU photo multiplier tube, an optical light guide and SCHOTT KV 540 filters. The PMT converts photons to an electrical signal that is digitized by an A to D converter. The computer software in the control system 136 may be written in LABVIEW and digitally filters the data and displays it as peak values or as a plot of voltage versus time.

In one embodiment, a motion device, represented by the arrows 138, is used to move the light source 110, accompanying optical elements 118, 114, 124 and the detector 138 relative to the fibers 104 and the support 102, or visa versa. The motion device 138 may be any suitable mechanism, such as a linear positioning robotic track system that can move along two or more axes. In one embodiment, the motion device 138 is the NEWPORT XY motorized stage with or without a micrometer Z stage. This allows light to be sequentially directed into a first end 132 of each of the fibers 104 of each bundle. Alternatively, the scanning mirror 114 can be moved to sequentially direct the light at one or more focusing lenses 118 above each fiber. In yet another embodiment, light may be directed simultaneously at the first end of all of the fibers in the bundle.

The control system 136 is coupled to the light source 110, the detector 126 and the motion device 138. The control system 110 controls the light source 110, the detector 126 and the motion device 138 and analyzes results obtained from the detector 126.

In use, a bundle of fibers 104 is inserted into a solution containing the mobile chemical species or target 108. The mobile chemical species or target 108 thereby contacts each immobilized chemical species or probe 106 on the surface of the fibers 104 in that bundle. Binding may then occur between complementary probe and target pairs.

The control system 136 then instructs the motion device 138 to move the light source 110 and/or the fibers 104 relative to one another so that light can be directed into a first end 132 of a first fiber. Alternatively, the control system 136 moves the scanning mirror 114 so that reflected light would be directed into a first end 132 of a first fiber. The control system 136 then activates the light source so that light rays 112 are directed at the scanning mirror 114 and/or focusing lens 118. The focusing lens may be a cylindrical lens that forms the rays 112 into a focal point at a fiber's first end 132. The focal point may form a plane perpendicular to the fiber's length so that the fiber and the light source do not require exact alignment.

When light is focused into a wave-guide, such as an optical fiber 104, the light enters at many different angles relative to the fiber's surface. Angles greater than the critical angle (θ) pass out of the fiber. Angles at or less than the critical angle (θ) internally reflect and stay inside the fiber. The critical angle is a function of the wavelength of light (λ), the index of refraction of the fiber (n₁) and the index of refraction of the outside material (n₂). In a wave-guide , n₁ is greater than n₂.

Since photons are not just particles but also waves, a portion of the internally reflected light passes to the surface of the optical fiber. Because the surrounding material (such as air) has a lower index of refraction, the outside portion of the wave moves faster than the portion of the wave remaining in the higher index fiber. Consequently, the wave starts to turn towards the fiber surface, eventually entering back into the fiber continuing a zigzag pattern through the fiber. The light extending outside the surface is know as an evanescent wave. Since the light is injected at many different angles, it reflects all along the fiber surface generating a uniformly distributed evanescent wave around the entire fiber surface. The amount of energy in this evanescent wave varies with conditions, but it can be as much as 3% of the total light in any axial cross section of the fiber. A general rule is that the evanescent wave extends about half the wavelength of light past the surface with intensity highest at the surface and dropping non-linearly with distance (D_{P}) from the surface. The exact equation is:
D_{P}=λ/4πsqrt(n₁²sin²(θ)−n₂²)

Commercial fibers are designed to transmit light with minimal optical loss over long distances despite being in contact with other fibers, clamps, tubes, etc. As a evanescent wave would bleed much of the light when in contact with a higher index material, commercially available fiber optics may have a cladding around the core fiber. This cladding is another transparent layer surrounding the fiber core that has a lower index of refraction than the core. Cladding also gives the core added mechanical strength. While cladding is essential for typical fiber purposes, it is unacceptable for the system of the present embodiment. Accordingly, the optical fibers may be custom manufactured without cladding, such as by using a heat and pull technique.

The light 112 passes internally from the first end 152 to the second end 134 of the fiber 104, where it is reflected off the reflective coating at the second end and returns back toward the light source 110. However, in the process of internal reflection, an evanescent wave 120 is created along the fiber's surface. The evanescent wave 120 extends beyond the fiber's surface at about half the wavelength of light to illuminate any labeled and bound target-probe pairs attached to the fiber's surface.

If the target 108 hybridizes to a probe 106, and it is labeled with a fluorescent molecule 130, the evanescent wave 120 causes fluorescent light 122 to be generated. The intensity of this evanescent wave 120 exponentially dissipates with distance from the surface of the fiber 104 and almost disappears beyond 300 nanometers from the fiber's surface. Therefore, only the fiber 104 and bound target-probe pair on the surface of the fiber are illuminated, i.e., the bulk material around and outside of the fiber is not illuminated. This improves the overall signal to noise ratio received by the detector 126.

The photons generated by exciting the labeled probe are focused by the focusing lens 124 and directed toward the detector 126. The detector records the exact location of the source of the fluorescent light to enable the control system 136 to later identify the immobilized chemical species or probe 106 to which the mobile chemical species or target 108 is bound, thereby identifying the target.

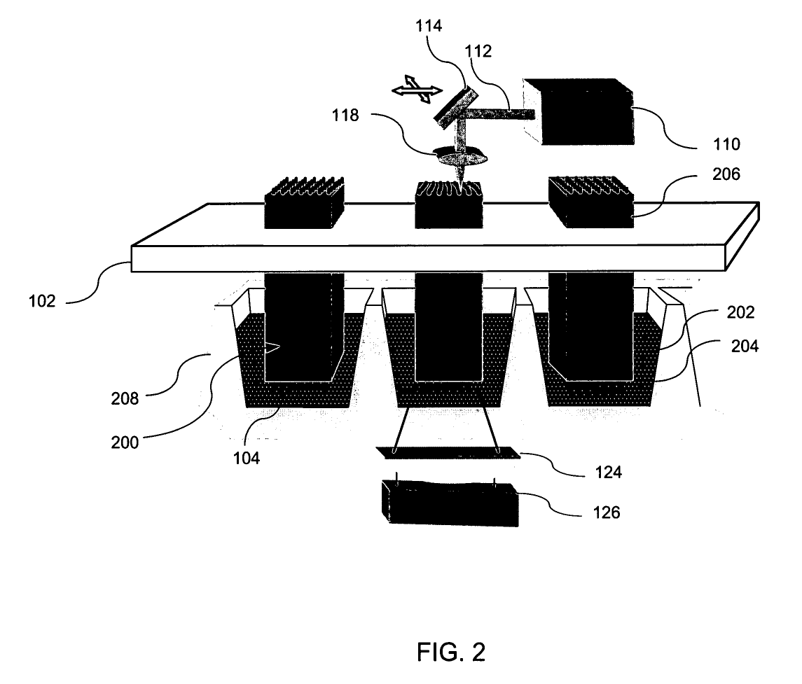

The control system 136 then instructs the motion device 138 to move the light source 110 and/or the fibers 104 relative to one another so that light can be directed into a first end 132 of the next fiber. This is repeated until light has been directed into all of the fibers. At the same time, the detector 126 is moved from fiber to fiber, or bundle to bundle, as described below in relation to FIG. 2 to detect fluorescence and to identify binding of targets and probes. Alternatively, light is directed at the first end of all optical fibers simultaneously in a particular bundle, and any binding is detected.

Because the selective illumination caused by the evanescent wave is only generated at the surface of each fiber, excess unreacted labeled species need not be removed before illumination. Of course, where desired, the excess unlabeled chemical species can be washed-away prior to illumination and detection.

An example of the use of the above described system will now be discussed. In a DNA hybridization application, a solution containing a target DNA fragment 108 labeled with a fluoraphore is placed into a well. A probe DNA fragment 106 is attached to the fiber, as explained above. If the structure of the target DNA fragment 108 matches the structure of the probe DNA fragment 106, the target DNA fragment 108 hybridizes with the probe DNA fragment 106 and remains at the fiber's surface. Since the evanescent wave 120 only illuminates near the fiber surface, the target DNA fragment labeled with the fluoraphore is illuminated or fluoresces when hybridized to a probe DNA fragment. Mismatched targets and probes will not hybridize and, therefore, will not fluoresce since they will not congregate near the fiber's surface. Thus, hybridization of the target DNA fragment to a particular probe DNA fragment is indicated by the presence of fluorescent light. If the interaction between the target DNA fragment and the probe DNA fragment causes an increase or decrease in the absorbance of a particular wavelength of light, the area around that fiber will emit either a greater or lesser quantity of light as compared with other fibers where no interaction occurs As the intensity of this evanescent wave exponentially dissipates with distance from the surface of the fiber, only the fibers that are illuminating relatively brightly are detected and recorded.

FIG. 2 is a partial oblique view of the system 100 for detecting the binding of chemical species shown in FIG. 1. As shown, multiple bundles 200 can be grouped on a support 102. Each bundle 200 may include multiple rows of stacked fibers at least partially encased in an optically opaque and chemically inert column 206. Each column 206 may be made from any suitable material, such as a polymer resin or the like. The bundles 200 are configured to be dipped into wells 204 formed in a plate 208, such as a standard 96 well plate. The plate may be constructed of an optically transparent material that allows the light generated by the light source 110 to pass there-through.

In use, each well may be filled with a different target solution 202 containing the mobile chemical species or target 108 (FIG. 1). While the fibers 104 are in the solution 202, the light beam 112 is sequentially directed into each fiber 104 of each bundle 200. When fluorescent light is generated, as described above, the light passes through the plate 208, through the lens 124 and into the detector 126. Alternatively, the detection may take place once the fibers 104 are removed from the solution.

FIG. 3A is a perspective view of a partially assembled bundle 302 of optical fibers 104 to be used in the system 100 shown in FIGS. 1 and 2. FIG. 3B is a perspective view of the assembled bundle 304 shown in FIG. 3A. As described above, each fiber 104 is initially coated with a different immobilized chemical species or probe 106 (FIG. 1) using any suitable system and method. Such fibers may be wound onto a reel and kept for subsequent use when making bundles for use in the system 100 (FIG. 1) of the present embodiment.

To manufacture a bundle 302, the fibers 104 are extended and laid onto a base 306. In one embodiment, the base 306 forms substantially parallel V-shaped depressions 308 extending along the entire length of one side of the base 306. The side of the base 306 opposite to the depressions may be flat or complementary to the fibers, e.g., concave depressions sized to at least partially receive the fibers. Each depression 308 is sized to receive a single fiber 104. It should, however, be appreciated that the depressions may be any suitable shape such as U-shaped or the like.

Once all of the depressions 308 in a base have received different fibers, another base 306 is placed on top of the base with the fibers thereon. The new base then receives optical fiber's in its depressions, and so on. In this way, layers of bases and substantially parallel fibers can be stacked one on top of the other until a bundle having the desired number of fibers has been constructed. Finally, a cap 312 is placed over the upper base 306 to fix the fibers in the upper base. In one embodiment, the fibers may be positioned on multiple bases 310 and then cut to form distinct bundles, as shown in FIG. 3A.

In one embodiment, the depressions 308 are configured and dimensioned so that once the bundle has been constructed, each fiber has some space around it to allow the mobile chemical species or target to flow along at least part of the length of the fiber but has a tight enough fit so that the fiber cannot move within the depressions 308. Alternatively, a predetermined length 314 of the fibers 104 may extend from the bases 306 allowing for contact with the mobile chemical species or target solution.

In addition, the second end 134 (FIG. 1) of each fiber may be coated with a reflective coating 312 to prevent light from escaping out of the fiber's second end. Furthermore, each fiber's first end 132 (FIG. 1) may be polished to be optically flat (substantially perpendicular to the fiber's longitudinal axis) so that light entering the first end is not reflected or scattered.

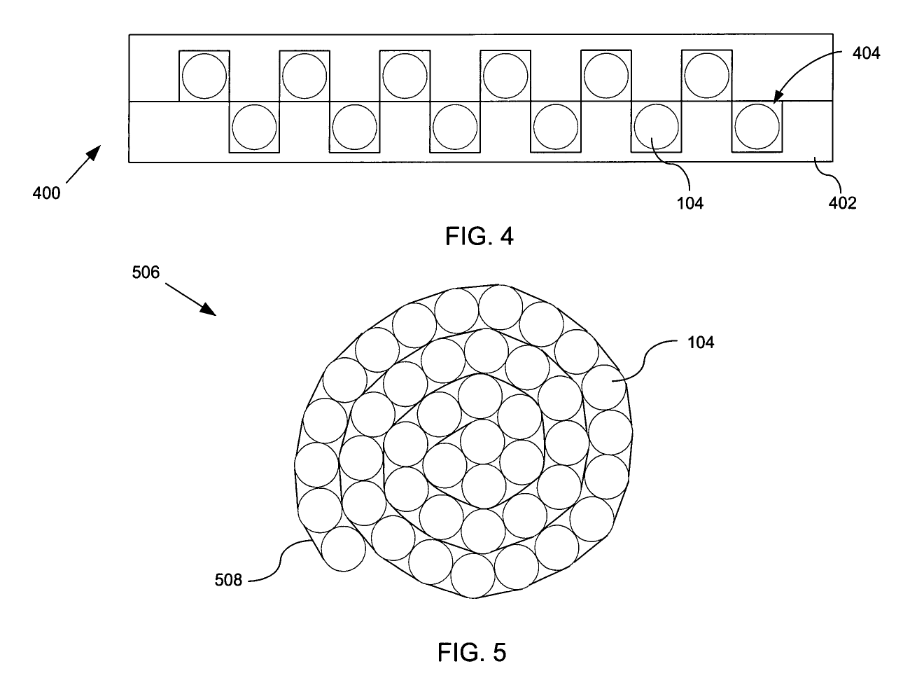

FIG. 4 is another embodiment of two rows 400 of bundles according to another embodiment of the invention. In this embodiment the base 402 includes multiple substantially parallel U-shaped depressions 404. A different fiber 104 is placed in each depression, and the bases are stacked one on top of the other such that the depressions face one another but are offset from one another. Additional bases and fibers may be stacked one on top of the other until the desired bundle is formed.

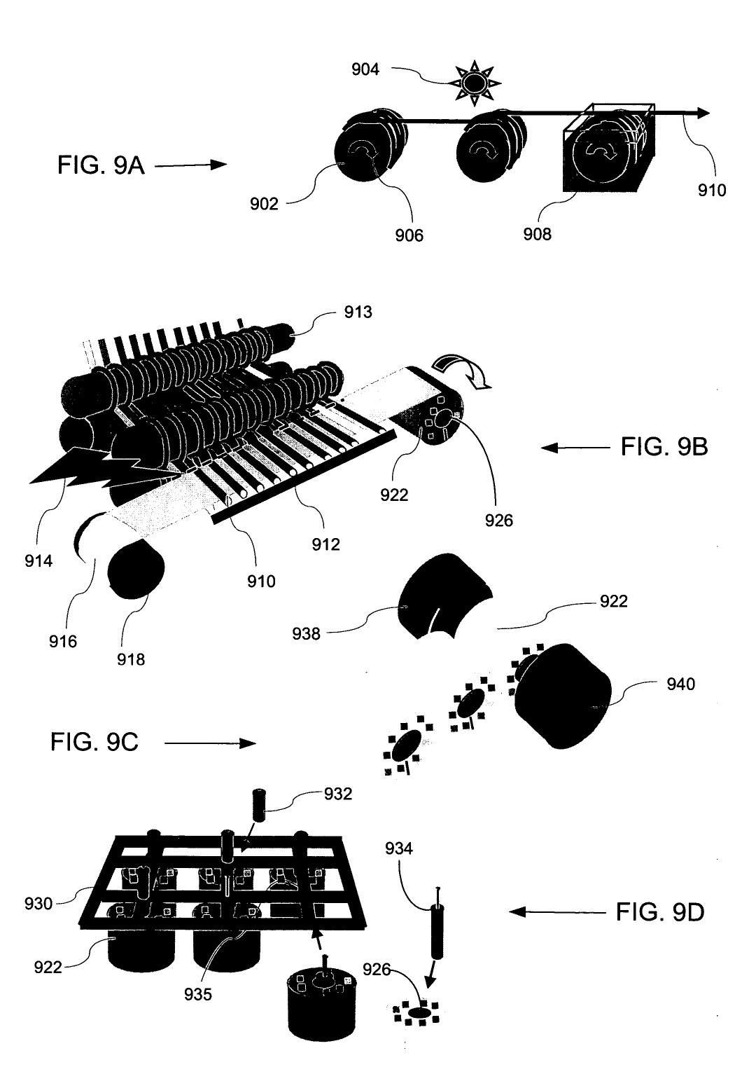

FIG. 5 is yet another embodiment of a bundle 506. Here, the fibers 104 are positioned next to each other on a thin adhesive film 508 such that the fibers form a mat of substantially parallel fibers 104. The mat of substantially parallel fibers is then rolled into a spiral bundle. In an alternative embodiment, the fibers may be randomly arranged in a bundle. A more detailed description of a process for manufacturing such a bundle 506 is described below in relation to FIGS. 9A-9D.

FIGS. 6A-6D are a flow chart of different methods for attaching probes to optical fibers and detecting binding of a probe with a target according to an embodiment of the invention. FIG. 6A is a block diagram of a multiple step workflow for constructing probes for use in an optical fiber bundle for detecting binding of chemical species. This workflow fundamentally consists of three steps. The first step 602 comprises an in-solution oligonucleotide ligation assay (OLA), which uses a pair of oligonucleotide probes (oligomers) that hybridize to adjacent segments of DNA including a variable base. Initially, one or more known sequences 604 are introduced into a solution in a mixing vessel 618, such as a test tube. The known sequence may be combinatorial DNA (cDNA), genomic DNA (gDNA), MRNA, or the like. Each known sequence 604 has a portion that is complementary to that of the probe sequence 640 that is ultimately required to be generated, and, therefore, is chosen accordingly.

Multiple oligonucleotides probes which are the probes desired to be ultimately attached to the fiber used for detecting binding, as described below in relation to FIG. 6D, are also added to the solution in the vessel 618. Some of the oligonucleotide probes 610(1), 610(2) and 610(3) have a zip code sequence (TSO-Zip) 612(1), 612(2) and 612(3) attached thereto, while other target specific oligonucleotide probes 606(1), 606(2) and 606(3) are labeled with a dye, Q-DOT, or the like (TSO-label) 608(1), 608(2) and 608(3). One should note that different oligonucleotide probes 610(1) and 610(2) have different zip code sequences 612(1), 612(2) and 612(3), and different oligonucleotide probes 606(1), 606(2) and 606(3) may have different labels attached thereto 608(1), 608(2), and (608(3), e.g., different colored dyes. Each OLA requires hybridization of the probes to a complementary portion of the known sequence 604. In other words, only probe 610(3) and 606(3) may hybridize with the complementary known sequence 604(3) at a specific location, but the others would not. The solution also includes ligation enzymes 614.

The solution is then heated to a hybridization temperature, such as 55° C., so that hybridization between the probes 610(1), 610(2), 610(3), 606(1), 606(2) and 606(3) and a known sequence 604(3) may potentially occur. Such hybridization is generally very sensitive to temperature, i.e., hybridization generally only occurs at an exact temperature. Once the probes are hybridized to the known sequence, the ligation enzymes 614 then covalently bond the probes together, forming a ligated probe sequence, as shown at step 602.

The temperature is then raised to “melt” each probe off a complementary known sequence 604, which then becomes available for another hybridization. Accordingly, thermal cycling may be used to effect linear amplification, thereby generating multiple ligated probe sequences 640. Furthermore, different known sequences 604, different probes 606(1), 606(2), 606(3), 610(1), 610(2) and 610(3), and different labels 608(1), may be provided in the same solution to generate multiple different probe sequences 640.

The second step 603 in FIG. 6A shows the addition of fibers 622 into the solution containing the probe sequences 640. The fibers 622 may be any suitable optical fibers, as described above. The fibers may be grouped together into a bundle in a fiber block 628, as shown, or may be bundled together later. In some embodiments, the bundle has a spacious circular pattern on one side thereof and a very compact arrangement the other side thereof, as shown in FIG. 6A. The number of fibers may vary from a few to several thousands with a single ring of fibers, multiple rings, or spiral arrangement of fibers, as described above.

The fibers 622 have universal probes 624 covalently attached thereto. The universal probes 624 all have the same Zip sequence (Fiber-Zip). Zip templates 616 are then added to the solution (or may have already been added in step 602). Each Zip template comprises: (1) a sequence complementary to both the Fiber-Zip of the probes 624 on each fiber 622; and (2) a sequence complementary to the TSO-Zip 612(1), 612(2) and 612(3) attached to each probe 610(1), 610(2) and 610(3), such that they can hybridize together. The Fiber-Zip and TSO-Zip can then covalently bond to each other by ligation using another ligation enzyme 620. Note, ligation enzymes 614 and 620 may be the same ligation enzyme. Accordingly, as the TSO-Zips 612(1), 612(2) and 612(3) have also been ligated to a TSO-label 608(1), 608(2) or 608(3) during the first ligation, the fiber 622 is now covalently attached to one or more probes each having a label attached thereto.

The Zip templates 616 used may have different TSO-Zip sequences for each Fiber-Zip sequence, enabling a multiplex of different probe sequences 640 to be attached to the fiber 622, i.e., ultimately, a single fiber may be used to detect more than one target. In this case, each different probe sequences 640 on a particular fiber may have a different unique label, such as different colors or the like, for implementing multiplexing. The above-described technique, therefore, only requires a single type of fiber with a single type of probe attached initially thereto. Accordingly, only one type of fiber needs to be manufactured, thereby simplifying quality control and costs. In some embodiments, the entire process for assembling the probe sequences 640 takes only a few minutes. Note, that there may be multiple different types of universal probes 624 attached to each fiber.

The third and final step 605 in FIG. 6A is a detection step, as described in more detail below in relation to FIG. 6D. Also note, that the fibers may be washed between steps 603 and 605. Table 1, below, is an example of some of the parameters for the method described above in relation to FIG. 6A.

TABLE 1

| | |
|---|---|
| Assays per run | 100 (e.g. 25 fibers * 4 colors) |
| Samples per fiber | 1 |
| block | |
| Fiber block size | 20 mm diameter * 20 mm tall |
| Fiber core diameter | 50 μm |
| Fiber jacket diameter | 100 μm |
| Fiber length (mm) | 20-30 |
| Sample volume: | <0.5 μL |
| Protocol | OLA on target, OLA product to |
| | fibers, read |
| Photon sensor | Photo Multiplier Tube or Avalanche |
| | Photo Diode |
| Run time | 2 hours, 1 hour per OLA reaction |

FIG. 6B is a block diagram of another multiple step workflow for constructing and using an optical fiber bundle for detecting binding of chemical species. Initially at step 642, a known sequence 604 is introduced into a solution in a mixing vessel 618, such as a test tube. The known sequence may be combinatorial DNA (cDNA), genomic DNA (gDNA), mRNA, or the like. The known sequence 604 has a complementary sequence to that of the probe sequence 640 that is ultimately required to be generated, and, therefore, is chosen accordingly.

Multiple target-specific oligonucleotides probes 650 are also added to the solution in the mixing vessel 618. These probes 650 may have different sequences, thereby providing different probes for use. Some of the target-specific oligonucleotide probes 650 have a zip code sequence (TSO-Zip) 612 attached thereto, while others are target specific oligonucleotide probes (TSO) 652 only. These target-specific oligonucleotide probes 650 and 652 are designed to hybridize sequentially on a particular known sequence 604 and ligate together, as described above in relation to FIG. 6A. Furthermore, the TSO-Zip 612 includes a sequence 654 for hybridization with a universal forward primer, and TSO 652 includes a sequence 656 for hybridization with a universal reverse primer (or vise versa).

At step 644, complementary universal forward primers 658 and reverse primers 660 as well as DNA polymerase (not shown) are added to the solution. The complementary reverse primers 660(or complementary forward primers 658) are labeled with a dye 608, Q-DOT, or the like. The probe sequence 640 of step 642 is amplified by polymerase chain reaction (PCR) to produce a copy of the same sequence having a label 608. Unligated probes will not amplify exponentially.

At step 646, fibers 622 are inserted into the solution. The fibers 622 have probes 624 covalently attached thereto. Each probe 624 has a Zip sequence (Fiber-Zip) designed to be complementary to the TSO-Zip 612 sequence. This allows the product to hybridize with the probe 624, thereby labeling the fiber 622, such as with a dye 608. The final step 648 is a detection step, which is described in more detail below in relation to FIG. 6D. Also note, that the fibers may be washed between steps 646 and 648.

FIG. 6C is a block diagram of another multiple step workflow for constructing and using an optical fiber bundle for detecting binding of chemical species. This embodiment does not require any zip codes and makes use of a phycoerythrin label. Initially, at step 662, universal probes 668 and ligation enzymes are mixed with the known sequence 604 in solution. The universal probes 668 are used to generate universal target specific oligonucleotides having a common-sequence of bases (depicted by “cccc”) and an all-combination sequence (depicted by “nnnnn”) for a set number of bases. The common sequence is selected for a particular application. For example, in expression analysis of human genes, a short 4-base sequence might be selected to ensure that each gene has that sequence at least once. A 3-base sequence might also work, but longer sequences of 5 or more would probably not be present in every gene.

To enable the short 4-base sequence to hybridize to the appropriate target, an all-combination sequence is added, as the short 4-base sequence of common bases alone are generally too short to hybridize strongly. For example, an all combination 5-mer sequence may be sufficient, increasing the target specific sequence to nine bases. For a 5-mer all-combination sequence, 1024 universal target oligonucleotides would need to be synthesized, each with a unique combination of the 5-mer but all having the same common 4-mer sequence at one end of the oligonucleotides.

All universal probes are then mixed together. The same mixture of universal probes may be used for any test. The manufacturing cost of making the mixture of universal probes is low, since the oligonucleotides are made and mixed in bulk.

The universal probes are mixed into a solution containing a known sequence 604. The known sequence 604 may be combinatorial DNA (cDNA), genomic DNA (gDNA), mRNA, or the like. The known sequence 604 has a complementary sequence to at least some of the universal probes that is ultimately required to be generated, and, therefore, is chosen accordingly. The solution also contains ligation enzymes 614.

At step 664, the fibers 662 are introduced to the sample mixture. On each fiber is a target specific sequence 624 that is designed to hybridize to a particular target sequence adjacent to a common base sequence such that a universal probe will hybridize and be ligated to it, thereby covalently binding the universal probe to the fiber when the right target is present. All universal probes are biotinylated, i.e., have a biotin 670 attached thereto. After ligation, the known sequence 604 is “melted” away from the probes and the fibers are washed, leaving only universal probes covalently attached to the fibers.

At step 666, a label that will bind to the biotin 670 on the fibers, is added to the solution. An example of such labels is phycoerythrin, a protein that is about 20 times brighter than most dyes, or streptavidin coated Q-DOTS. As before, the final step 666 is a detection step, and is described in more detail below in relation to FIG. 6D. Also note, that the fibers may be washed between steps 664 and 666. Table 2 gives an example system specification for FIG. 6C.

TABLE 2

| | |
|---|---|
| Assays per run | 10000 (e.g. 10,000 fibers * 1 color) |
| Samples fiber block | 1 |
| Fiber per block size | 200 mm diameter * 200 mm tall |
| Fiber core diameter | 50 μm |
| Fiber jacket diameter | 100 μm |
| Fiber length | 200-300 mm |
| Sample volume: | 200 μL |
| Protocol | OLA on target, OLA product to |
| | fibers, read |
| Photon sensor | Photo Multiplier Tube or Avalanche |
| | Photo Diode |
| Run time | 2 hours, 1 hour per OLA reaction |

FIG. 6D is an oblique view of a system 670 for detecting the binding of chemical species. The system 670 includes a support 676 and a fiber block 628 for affixing the elongate optical fibers 622 relative to one another. The optical fibers 622 may be any fibers made as described herein and have a first end 680 and a second end (hidden) remote from the first end. The optical fibers 622 are flexible.

The support 676 may be a planar disk that is configured to be rotated about a central axis 682, as shown by the arrow. The fiber block 628 may be any suitable shape and is configured to rotate around the same central axis 682, together with the support 676. In some embodiments, the support 676 and the fiber block are the same integral component. In some embodiments, the fiber block 628 may be made from a resin, a wax or the like.

The support 676 securely positions the fibers 622 in a ring near their first ends 680, such that the fibers near their first ends 680 are substantially parallel to one another and substantially perpendicular to the support 676. Alternatively, the support 676 may position the fibers in any arrangement, such as in a spiral arrangement, as long as the fibers are sufficiently spaced apart so that light entering each fiber's first end 680 does not substantially enter an adjacent fiber's first end. In some embodiments, a mask oriented above the fibers' first ends 680(not shown) having an aperture sized to only allow light to reach a single fiber at a time may be used.

The fiber block 628 securely positions the fibers 622 in any suitable layout, such as a circular layout, a matrix layout, a spiral layout, or the like. The layout of the fibers at their second ends, i.e., remote from their first ends 680, occupies much less cross-sectional area than the layout of the fibers at the support 676. That is, the second ends of the fibers are much closer to one another than the fibers' first ends 680. Although the fibers are located much closer to one another at their second ends for easier detection, they too remain substantially parallel to one another near their second ends.

The system 670 also includes a well 684, a light source 672, a first lens 674, a second lens 686 and a detector 688, all of which are similar to the corresponding components described above in relation to FIG. 1. These components are fixed in space relative to the support 676, fiber block 628 and the fibers 622.

In use, the end of the fibers are inserted into the well 684 containing a sample solution. The support 676,fiber block 628 and the fibers 622 are rotated to align a first end 680 of a first fiber with light exiting the light source 672. The light is focused into a focal point at or near the first fiber's first end 680 by the first lens 674. The light then forms an evanescent wave near the circumference of the fibers. This light may excite the labels attached to the probes attached to the fibers, if binding occurred between the probes constructed on the fibers and target present in the sample solution. Any light emitted by the labels is focused by the second lens 686 into the detector 688, which then detects whether binding has occurred. Further details of the use of a similar system can be found above in relation to FIG. 1.

Furthermore, in some embodiments, the probes can be made on the fibers using in-situ synthesis techniques. Also, in some embodiments, the above-described system 670 requires only a very small amount of sample for a multiplex of assays. The system is also a low-cost instrument, requiring only one motor, simple optics, a photo detector, and laser. The system is compact and simple to use, where an operator adds the target sample and waits for an answer. Also, the number of different probes in free solution is small (1024), enabling a very large multiplex of assays per test.

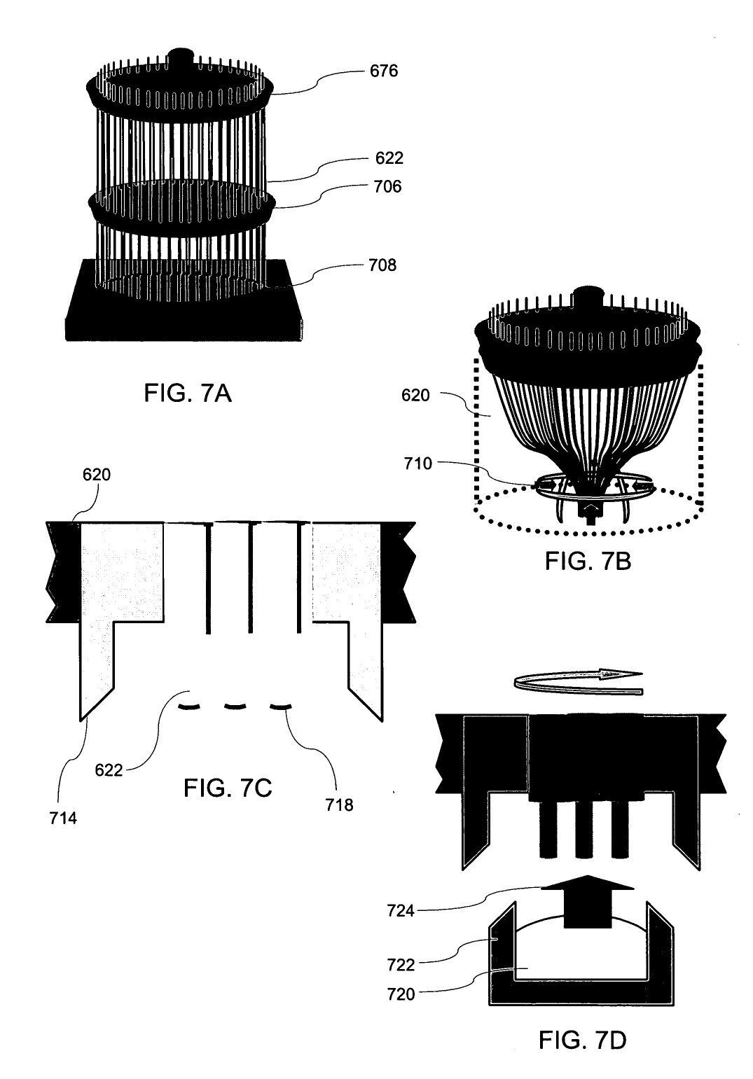

FIGS. 7A-7D are oblique views of the various stages of assembly of the system 670 (FIG. 6D). As shown in FIG. 7A, the fibers 622 are arranged parallel to one another in a ring. The support 676 affixes the fibers parallel to one another in a ring near the fibers' first ends. An additional support 706 may be used to add additional support to the fibers. The additional support 706 has holes therein, through which the fibers pass. The additional support 706 can be lowered to separate the fiber tips or raised to enable the fiber tips to be compressed together. In this configuration, each fiber can be inserted into its own well such that one or more probes 708 may be attached to the second end of each fiber. For example, each well contains the chemistries necessary to permanently attach a unique oligonucleotide probe to a corresponding fiber, for example, as described above in connection with FIGS. 6A-6C.

FIG. 7B shows how the fibers are bundled into a compact arrangement. The additional support 706 is raised adjacent to the support 676. In some embodiments, an automated mechanism then pushes the second ends of the fibers near one another, as shown by arrows 710. The fiber block 628 is then formed around the fibers 622. For example, a wax or resin can be poured around the fibers and cured to form the fiber block 628.

FIG. 7C shows how the fibers are inserted into a collar 714. The collar 714 bundles the fibers 622 close to one another near the fibers' second ends. The collar 714 is fixed within the fiber block 628 material to provide rigidity to the assembly. The collar 714 may have a shape complementary to a target tube described below. The end of each fiber may be coated with a reflective material 718.

FIG. 7D shows a target tube 722 containing a sample solution 720 comprising targets. The target tube 722 may have a shape complementary to the collar 714 (FIG. 7C). The target tube 722 is pushed into contact with the collar 714 (Figure C) as shown by the arrow 724. This allows the sample solution 720 to contact the fibers 622 (FIG. 7A). In some embodiments, the target tube is made from an optically transparent material. In general, the number and diameter of fibers determines the minimum sample solution volume. For example, a 100-assay block with 25 fibers at 100 μm diameter and 4 colors per fiber could require as little as 0.5 μL of target. Light is then directed down the fibers and the collar and fibers rotated, as described above in relation to FIG. 6D.

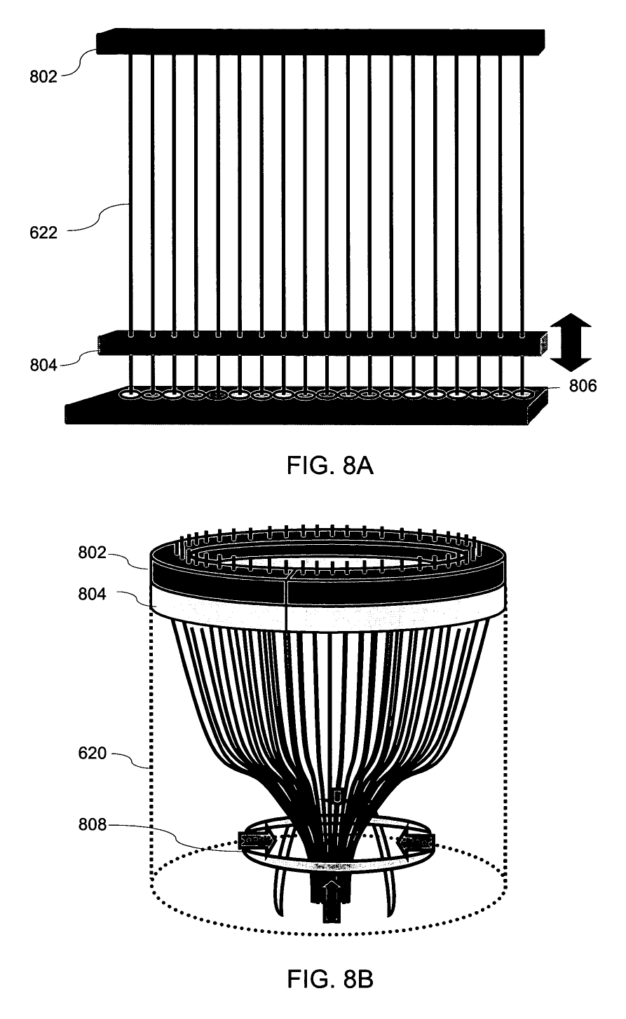

FIGS. 8A and 8B show another way to construct the system. In particular, FIG. 8A a linear arrangement of fibers 622 and wells 806. A flexible band 802 supports the fibers in a fixed arrangement parallel to one another. A second flexible band 804 with through holes can move close to or further away from the support band 802 such that the second ends of the fibers can be spaced far apart to fit into wells or raised to enable the fiber tips to be compressed close together. As described above, each well contains the chemistries necessary to permanently attach a unique probe to that fiber. After attaching the probes to the fibers, the bands can be flexed into circles or spirals, as shown in FIG. 8B. The second ends of the fibers 622 can then be clamped together, as shown by reference numeral 808, and the fibers affixed in the fiber block 628, in a similar manner to that described above in relation to FIG. 7B.

FIGS. 9A-9D are oblique views of a system for making multiple bundles of optical fibers to be used in a system for detecting the binding of chemical species, according to an embodiment of the invention. FIG. 9A shows the system for depositing a chemical species 908 onto an optical fiber 910. The bare optical fiber is unrolled from a reel 902 as shown by arrow 906. The optical fiber 910 is then plasma treated 904 to facilitate binding of a chemical species 908, which may include the species necessary to construct probes on the fibers, with the optical fiber 910. The chemical species 908 is then deposited on the optical fiber 910. The chemical species 908 may be deposited on the optical fiber 910 by any suitable means such as by passing the optical fiber through a bath of the chemical species 908, as shown, wicking the chemical species onto the optical fiber, spraying the chemical species onto the optical fiber, or the like. Each optical fiber that will eventually be bundled together may receive one or more different immobilized chemical species thereon.

FIG. 9B shows the system for bundling the optical fibers 910. Multiple optical fibers 910, each having a different chemical species thereon, is unwound and placed onto tape 916. The optical fibers may be arranged substantially parallel to one another and substantially perpendicular to the longitudinal axis of the tape 916. The tape 916 may have an adhesive on the surface thereof that faces the optical fibers 910. This adhesive constrains the optical fibers in contact with the tape and prevents them from contacting one another. The optical fibers 910 are then cut, as depicted by the cutting symbol 914, such that a short length of each optical fiber extends from, and overlaps at, the sides of the tape 916. Furthermore, a step 912 may be used to ensure that the ends of the fibers are aligned.

The tape 916 may then be wound from a roll of tape 918 onto a spool 926 until enough tape is exposed for the next set of optical fibers to be unreeled onto the tape and the process repeated. This results in consecutive spiral layers of tape and optical fibers that are wound onto the spool 926 to form a bundle 922 of optical fibers.

FIG. 9C shows the system for polishing and coating the bundles. Each bundle 922 has multiple optical fibers extending from each longitudinal end. The end of the optical fibers that receives light from the light source is polished by a polisher 938 to eliminate any ragged ends and create a substantially optically flat surface that is substantially perpendicular to each optical fiber's longitudinal axis. The substantially flat surfaces prevent scattering of the light entering the optical fiber. The opposite end of each fiber is coated with a reflective coating, such as by contacting a reflective-ink-pad 940 against one side of the bundle.

FIG. 9D shows the system for creating an array of bundles 922. Once multiple bundles 922 have been formed, a shaft 934 is placed through the spool 926 of each bundle 922. The shaft 934 may have two longitudinal sections of different diameters. The first longitudinal section is sized to tightly fit within the spool 926, while the second longitudinal section is sized to fit through holes 935 formed in a plate or frame 930. An end cap 932 is then affixed to the second longitudinal section to securely couple the bundle to the frame 930, while still allowing the bundle to be rotated about the shaft 934.

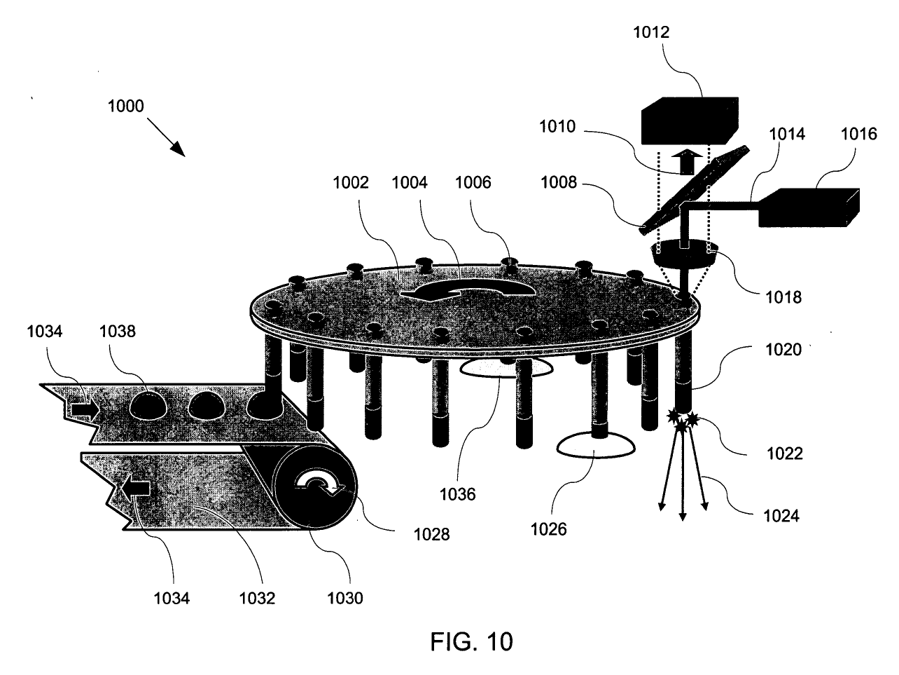

FIG. 10 is an oblique view of yet another system 1000 for detecting the binding of chemical species. This system 1000 is similar to the system described above in relation to FIG. 6. Optical fibers 1006 are coupled to support disk 1002, such that the optical fibers 1006 extend through the support disk 1002 substantially parallel to one another in an annular configuration. The support disk 1002 is rotatable about its central longitudinal axis as depicted by arrow 1004. The support disk may be rotated by a stepper motor (not shown) that rotates the support disk in discrete steps rather than with a continuous movement. In a similar manner to that described above, each optical fiber 1006 has one or more chemical species or probes 1020 immobilized or covalently attached thereto.

The system 1000 also includes a conveyer 1032 that is driven by a rotating drum 1030. The rotating drum 1030 is rotated in the direction depicted by arrow 1028, thereby causing the conveyer 1032 to move in the direction depicted by arrows 1034. The rotating drum 1030 may be rotated by another stepper motor that rotates the rotating drum in discrete steps rather than with a continuous movement. The conveyer 1032 is configured to transport a mobile chemical species or target solution 1038 towards the optical fibers 1006. In one embodiment, individual drops of the target solution 1038 are disposed on the conveyer 1032 at a predetermined distance from one another.

The system 1000 further includes a light source 1016 and detector 1012 similar to those described above. Optical elements, such as one or more lenses 1018, may be used to focus light from the light source into each optical fiber or to focus light emitted from the circumference of the optical fiber into the detector 1012. A dichroic mirror 1008 may be positioned between the light source 1016, the optical fibers 1018 and the detector 1012. The dichroic mirror 1008 is a special type of interference filter that selectively reflects light according to its wavelength or spectrum while transmitting the remainder of the light. Accordingly, the dichroic mirror 1008 reflects light from the light source at an end of each of the optical fibers 1006 but allows light generated by an evanescent wave at the circumference of the optical fibers to pass through to the detector 1010, as shown by arrow 1010.

As the support disk 1002 and the conveyer 1032 are rotated, a second end of each optical fiber 1006 touches a different drop of the target solution 1038. This allows each drop of target solution to at least partially contact a corresponding optical fiber. The support disk 1002 continues to rotate giving the target in the a target solution time to bind to the probe 1020. Any excess target solution that has not bound to the probe is then washed away from the optical fibers by passing the second end of the optical fibers through a wash solution 1026.

The light source 1016 is then energized such that a beam of light 1014 is directed at a first end of an optical fiber remote from the second end of the optical fiber. If binding occurred between a probe-target pair, an evanescent wave formed at the circumference of the optical fiber will cause the bound-pair to fluoresce or emit light. This emitted light is transmitted through the dichroic mirror 1008 and into the detector 1012. The support disk 1002 and the conveyor are then rotated. The fiber may again be washed at step 1036 to remove any hybridized target therefrom, e.g., the fiber is subjected to a heat wash solution to denature or remove the florescent target. In this way the same fiber may then be used again with another target. The process is then repeated, until all of the optical fibers have been subjected to the detection step. In this way, the detector 1012 can automatically detect the binding of chemical species on the surface of the optical fibers.

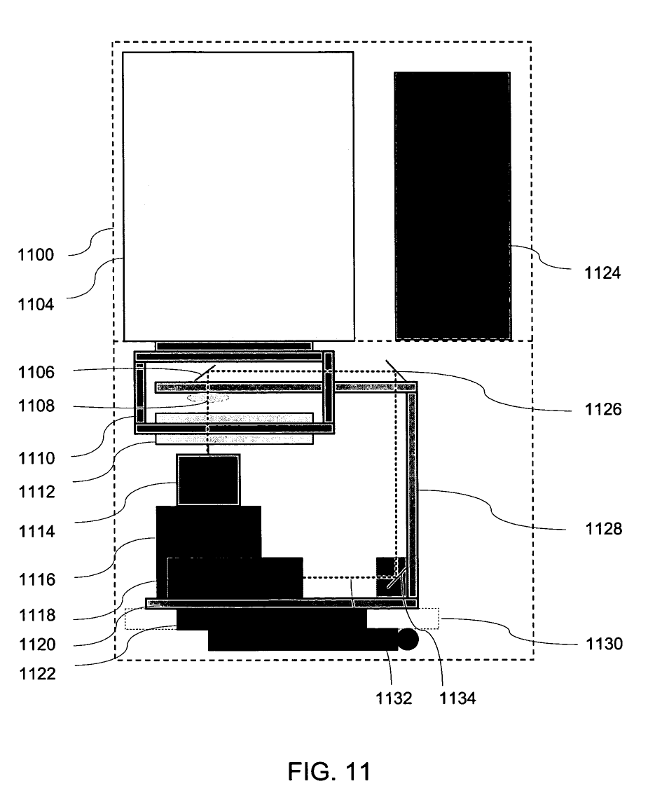

FIG. 11 is a side view of another system 1100 for detecting the binding of chemical species. The system 1100 includes a stationary block heater 1104 coupled to a heater frame 1110. The heater frame 1110 is configured to securely hold an assay 1112 that includes a support plate, bundle(s) of optical fibers, wells etc., similar to those described above. The heater frame 1110 is also configured to transfer heat from the block heater 1104 to the assay 1112 if heat is required, such as during a hybridization incubation step. However, it should be appreciated that if heat is not required, then the block heater may be eliminated from the system 1102, and the frame 1110 may be securely mounted to a stationary or immobilized object, such as by being bolted to a wall or larger frame bolted to the ground.

As with the systems described in relation to FIGS. 1-4B above, the system 1100 also includes a detector 1116 and a light source 1118, such as a laser. The system 1100 may also include optical elements 1114 for focusing light emitted from the assay 1112 into the detector 1116. The detector 1116, light source 1118 and optical elements 1114 are mounted, either directly or indirectly, on a XY platform 1120. The XY platform 1120 is coupled to a motion device 1122, such as an XY motor. The motion device translates the platform along a X axis (parallel to the page of FIG. 11) and along a Y axis (perpendicular to the page of FIG. 11). For example, the motion device 1122 can move the platform 1120 to the positions shown by the broken line 1130. The motion device 1122 may be any suitable motion device for translating the platform 1120 along at least one plane. Furthermore, the motion device may be controlled by a control system 1124 electrically coupled to the motion device 1122.

A C-shaped frame 1128 is also coupled to the platform 1120,and, accordingly, is free to move in the XY plane together with the platform 1120. Multiple mirrors 1134, 1126 and 1106 are also coupled to the platform 1120. These mirrors direct light emitted from the light source 1118 toward an end of each of the optical fibers. In one embodiment, these mirrors may be scanning mirrors. In another embodiment, only the mirror 1106 that is proximate to the ends of the fibers is a scanning mirror for sequentially directing light at each end of the optical fibers in a bundle of the assay 1112.

In one embodiment, optical elements, such as a lens 1108, may be positioned along the path of light emitted from the light source 1118 between the light source and the assay 1112. These optical elements may be used for focusing or conditioning the light before it is directed at an optical flow, and may include lenses, filters, or the like.

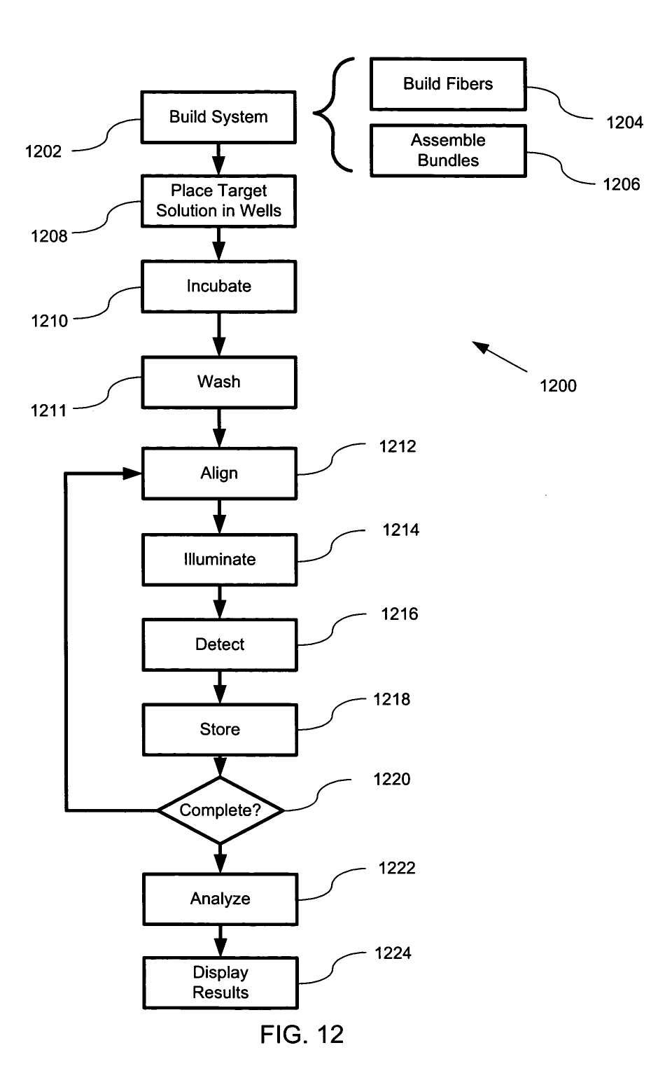

FIG. 12 is a flow chart of a method 1200 for detecting the binding of chemical species, according to an embodiment of the invention. The system, such as system 100 (FIG. 1), 600 (FIG. 6) or 800 (FIG. 8), is initially built at step 1202. To build the system, fibers with immobilized chemical species or probes thereon must first be manufactured at step 1204, an example of which is described in relation to FIG. 7A. A suitable method for manufacturing such fibers is disclosed in U.S. Pat. No. 6,1273,0812, which is incorporated herein by reference in its entirety. The fibers are then assembled into bundles, at step 1206, as described above in relation to FIGS. 3, 4A, and 4B and FIGS. 7B-7D.

A target solution 202 (FIG. 2) containing a mobile chemical species or target is then placed into the various wells, such as wells 204 (FIG. 2) or 618 (FIG. 6), or on the conveyor 832 (FIG. 8) at step 1208. The fibers and target solution may then be incubated, at step 1210, to aid binding or hybridization between the probes and target. After binding/hybridization has occurred, the fibers may be washed at step 1211. A first end of the optical fiber is then aligned with a path of light exiting the light source, such as light source 110 (FIG. 1), 1218 (FIG. 12), 602 (FIG. 6) or 816 (FIG. 8), and any intermediate optical elements. For example, the scanning mirror 114 (FIG. 1) redirects light at a first end of a first fiber. In another embodiment, the motion device 138 (FIG. 1) moves the light source, optical elements, and fibers relative to one another until the light path is aligned with the first end of the first fiber. In another example, the first support 606 (FIG. 6) is rotated about its central axis until the first end 616 (FIG. 6) of a first fiber is aligned with the light path (also see FIG. 8).

A control system then energizes the light source to illuminate the first end of the first fiber at step 1214. An evanescent wave is formed on the surface of the fiber by the light inside the first fiber. If binding occurred between any probe and target at the surface of a fiber, the label is excited by the evanescent wave, thereby illuminating the label and causing it to fluoresce. The detector, such as the detector 126 (FIG. 1), 1216 (FIG. 12), 614 (FIG. 6) or 812 (FIG. 8), then detects any such florescence at step 1216. The location of the detected florescence is then stored in the control system, at step 1218, for later analysis.

The control system then determines whether all fibers in that bundle (or system) have been illuminated and/or detected, at step 1220, i.e., whether the illumination and/or detection has been completed. If the illumination and/or detection has not been completed (1220—No), then the control system aligns the light path with the next fiber's first end. This continues until all fibers have been illuminated. Once all fibers have been illuminated and the session is complete (step 1220—Yes), the stored results of any detection are analyzed, at step 1222, and displayed to an operator of the system at step 1224.

The foregoing descriptions of specific embodiments of the present invention are presented for purposes of illustration and description. For example, any methods described herein are merely examples intended to illustrate one way of performing the invention. They are not intended to be exhaustive or to limit the invention to the precise forms disclosed. Obviously many modifications and variations are possible in view of the above teachings. For example, the sequencing by hybridization may be format I, II, or III. Also, any figures described herein are not drawn to scale. The embodiments were chosen and described in order to best explain the principles of the invention and its practical applications, to thereby enable others skilled in the art to best utilize the invention and various embodiments with various modifications as are suited to the particular use contemplated. Furthermore, the order of steps in the method are not necessarily intended to occur in the sequence laid out. Please note that aspects of the present teachings may be further understood in light of the examples described above, which should not be construed as limiting the scope of the present invention. It is intended that the scope of the invention be defined by the following claims and their equivalents.

1. A system for detecting binding of two chemical species, comprising:
a bundle of elongate optical fibers, each having a first end remote from a second end;
multiple probes, each attached to one of said optical fibers within a predetermined section between each of said optical fiber's first end and second end;
a well configured to hold a solution comprising a target and to receive at least said predetermined section of each of said optical fibers;
a light source configured to direct light into said first end of each of said optical fibers; and
a detector configured to detect light emitted by the binding of said target to at least one of said multiple probes.

2. The system of claim 1, further comprising multiple bundles and multiple wells.

3. The system of claim 1, wherein said detector is disposed in proximity to said second end of at least one of said optical fibers.

4. The system of claim 1, wherein said light source is disposed in proximity to said first end of at least one of said optical fibers.

5. The system of claim 1, wherein each of said wells is configured and dimensioned to receive a length of said bundle therein, and where a cross-section of said well is larger than a cross-section of said bundle.

6. The system of claim 1, wherein said optical fibers in said bundle are substantially parallel to one another.

7. The system of claim 1, wherein said optical fibers in said bundle are parallel to one another.

8. The system of claim 1, wherein said second ends of each of said optical fibers are coated with a reflective coating.

9. The system of claim 1, wherein said bundle comprises multiple adjacent layers of parallel optical fibers.

10. The system of claim 1, wherein said bundle comprises a layer of parallel optical fibers rolled into a cylinder.

11. The system of claim 1, wherein said bundle comprises multiple concentric layers of parallel optical fibers forming a cylinder.

12. The system of claim 1, wherein said bundle comprises a spiral layer of parallel optical fibers.

13. The system of claim 1, wherein said bundle comprises substantially parallel fibers forming a ring near said first ends and a bundle near said second ends.

14. The system of claim 1, wherein said bundle comprises parallel fibers forming a ring at said first ends and a bundle as said second ends.

15. The system of claim 1, wherein said light source is configured to generate an evanescent wave about a circumference of each of said optical fibers.

16. The system of claim 1, wherein predetermined lengths of said optical fibers near said first ends and said second ends are substantially parallel to one another.

17. The system of claim 1, wherein predetermined lengths of said optical fibers near said first ends and said second ends are parallel to one another.

18. The system of claim 1, wherein predetermined lengths near said first ends are substantially parallel to one another.

19. The system of claim 1, wherein predetermined lengths near said first ends are parallel to one another.

20. The system of claim 1, wherein predetermined lengths near said second ends are substantially parallel to one another.

21. The system of claim 1, wherein predetermined lengths near said second ends are parallel to one another.

22. The system of claim 1, wherein said first ends of said optical fibers are spaced further apart from one another than said second ends of said optical fibers.

23. The system of claim 1, wherein said predetermined section is near said optical fibers' second ends, and wherein a diameter of said bundle at said predetermined section is smaller than a diameter of said well.

24. The system of claim 1, further comprising a motion device for sequentially directing light from said light source into each of said optical fibers.

25. The system of claim 24, further comprising a control system for controlling said motion device.

26. The system of claim 1, further comprising a motion device for sequentially positioning said detector adjacent an illuminated optical fiber.

27. The system of claim 26, further comprising a control system for controlling said motion device.

28. The system of claim 1, wherein said light source is an excitation laser or an arc lamp.

29. The system of claim 1, wherein said detector is photon multiplier tube.

30. The system of claim 8, wherein said reflective coating is made from a metal.

31. A system for detecting binding of two chemical species, comprising:
multiple bundles of elongated optical fibers, where each optical fiber has a first end remote from a second end;
multiple probes, each attached to one of said optical fibers within a predetermined section between said optical fiber's first and second ends;
multiple wells, each configured to hold a solution comprising a target and to receive at least said predetermined section of each of said optical fibers of at least one of said multiple bundles;
a light source configured to direct light into said first end of each of said optical fibers; and
a detector configured to detect light emitted by the binding of said target to at least one of said multiple probes.

32. A system for detecting binding of two chemical species, comprising:
a bundle of elongated optical fibers comprising first ends remote from second ends, wherein said first ends are spaced further apart from one another than said second ends, and wherein predetermined lengths of said optical fibers near said first ends are substantially parallel to one another and predetermined lengths of said optical fibers near said second ends are substantially parallel to one another;
multiple probes, each attached to one of said optical fibers within a predetermined section between said first and second ends.

33. A method for detecting binding of two chemical species, comprising:
contacting a target with multiple probes each attached to a different elongated optical fiber of a bundle of elongated optical fibers between a first end and a second end of each of said optical fibers;
directing light at said first end of each of said optical fibers;
detecting at said second end of each of said optical fibers light emitted by the binding of said target to at least one of said multiple probes.

34. The method of claim 33, further comprising, before said contacting, attaching said probes to said optical fibers.

35. The method of claim 33, wherein said contacting comprises dipping said bundle into a solution congaing said target therein.

36. The method of claim 35, further comprising, before said contacting, placing target solution into a well.

37. The method of claim 33, wherein said detecting further comprises identifying those optical fibers that emit the most light.

38. The method of claim 37, wherein said identifying further comprises detecting those optical fibers that emit fluoresce the most.

39. The method of claim 33, wherein said directing further comprises forming an evanescent wave near a surface of each fiber.

40. The method of claim 33, further comprising separately directing and detecting for each optical fiber in said bundle.

41. The method of claim 33, further comprising directing and detecting for all optical fiber in said bundle simultaneously.

42. The method of claim 33, further comprising, prior to said contacting, forming a bundle of said optical fibers by stacking parallel layers of optical fibers adjacent to one another, rolling a layer of parallel optical fibers into a spiral, forming concentric rings of parallel sheets of optical fibers, or a bundle of randomly oriented fibers.

43. A method for making an optical fiber having known probes attached thereto, comprising:
providing a known sequence in a solution;
inserting a first probe having a zip code sequence (TSO-Zip) attached thereto into said solution;
inserting a second probe that is labeled into said solution;
allowing said first and second probes to hybridize with said known sequence;
adding first and second ligation enzymes into said solution;
allowing said first and second probes to covalently bond to each other using said first ligation enzyme to form a ligated probe sequence;
removing said ligated probe sequence from said known sequence;
inserting a fiber into said solution, wherein said fiber has a third probe attached thereto;
inserting a Zip template into said solution, wherein said Zip template is configured to hybridize to both said TSO-Zip and said third probe;
allowing said TSO-Zip and said third probe to covalently bond to each another using said second ligation enzyme; and
removing said TSO-Zip and said third probe from said Zip template sequence.

44. A method for making an optical fiber having known probes attached thereto, comprising:
providing a known sequence in a solution;
inserting a first probe into said solution, wherein said first probe has a zip code sequence (TSO-Zip) attached thereto and a sequence for hybridization with a universal forward primer attached to said TSO-Zip;
adding a second probe into said solution, wherein said second probe is attached to a sequence for hybridization with a universal reverse primer;
adding a forward primer to said solution;
adding a reverse primer to said solution, wherein said reverse primer is labeled;
adding polymerase to said solution;
allowing said first and second probes to hybridize with said known sequence;
inserting a ligation enzyme into said solution;
allowing said first and second probes to covalently bond to each other using said ligation enzyme to form a ligated probe sequence;
removing said ligated probe sequence from said known sequence;
amplifying said ligated probe sequence using said forward primer, said reverse primer, said polymerase and said ligated probe sequence through a polymerase chain reaction (PCR) technique;
inserting a fiber into said solution, wherein said fiber has a third probe attached thereto; and
allowing said TSO-Zip and said third probe to hybridize to one another.

45. A method for making an optical fiber having known probes attached thereto, comprising:
providing a known sequence in a solution;
inserting a first probe into said solution, wherein at least part of said first probe has a sequence that will hybridize with a portion of said known sequence, and wherein said probe has a biotin attached thereto;
inserting a ligation enzyme into said solution;
inserting a fiber into said solution, wherein said fiber has a second probe attached thereto, wherein at least part of said second probe has a sequence that will hybridize with a portion of said known sequence;
allowing said first probe and said second probe to hybridize to said known target;
allowing said first and second probes to covalently bond to each other using said ligation enzyme to form a ligated probe sequence;
removing said ligated probe sequence from said known sequence; and
attaching a label to said biotin.
